(19) United States
(12) Patent Application Publication
(10) Pub. No.: US 2020/0060640 A1
(43) Pub. Date: Feb. 27, 2020

(54) PATIENT TABLE DESIGN WITH REDUCED ATTENUATION FOR EMISSION AND TRANSMITTION TOMOGRAPHY

(71) Applicant: KONINKLIJKE PHILIPS N.V., HIGH TECH CAMPUS 5 (NL)

(72) Inventors: Andriy ANDREYEV, WILLOUGHBY HILLS, OH (US); Chuanyong BAI, SOLON, OH (US); Douglas B. MCKNIGHT, CHARDON, OH (US)

(73) Assignee: KONINKLIJKE PHILIPS N.V., HIGH TECH CAMPUS 5 (NL)

(21) Appl. No.: 16/609,666

(22) PCT Filed: May 1, 2018
(86) PCT No.: PCT/EP2018/061079

§ 371 Date: Oct. 30, 2019

Related U.S. Application Data
(60) Provisional application No. 62/492,341, filed on May 1, 2017.

(51) Int. Cl. A61B 6/00; A61B 6/04; A61B 6/03

(52) U.S. Cl. CPC A61B 6/547; A61B 6/0471; A61B 6/037; A61B 6/5276; A61B 6/035; A61B 6/0457

(57) ABSTRACT

A medical imaging subject support table includes a belt conveyor system with a conveyor belt (18) maintained in tension and passing through a bore (14) of an imaging device (12); and motorized pulleys (20) disposed at opposite ends of the bore to move the conveyor belt through the bore. Table supports (24) are positioned outside of the bore of the imaging device on opposite ends of the bore and support the conveyor belt outside the bore of the imaging device.

### FIELD

The following relates generally to the medical imaging arts, image positioning arts, image motion correction arts, and related arts.

### BACKGROUND

In most emission and transmission tomography scans, including positron emission tomography (PET), computed tomography (CT), or single photon emission computed tomography (SPECT), the patient is placed on a supporting transportation device (known as patient table) for the duration of the scan. The table carries the patient through the gantry or gantries (in the case of multi-modality) of the imaging device to ensure that all target volumes are being imaged. For imaging, the radiation either traversing the patient (e.g. in transmission CT) or originating from the patient (e.g. in PET, SPECT, and the like) preferably reaches the detectors without substantial radiation-attenuating obstacles. The table is one attenuating obstacle of concern in this regard. Attenuation caused by some existing commercially marketed patient tables is usually about 10%, and even more in other modalities such as in SPECT or CT (where lower energy particles are used). To compensate for the table attenuation, the injected dose to the patient can be increased (leading to undesirably higher radiation exposure to the patient) or the scan duration can be increased, undesirably reducing workflow throughput.

Attempts have been made to make the table thinner and/or use less dense materials, however, in that case the table may become too flexible and lead to table deflection or sagging, which can introduce undesirable motion errors in the imaging data.

The following discloses new and improved systems and methods to overcome these problems.

### SUMMARY

In one disclosed aspect, a medical imaging subject support table includes a belt conveyor system with a conveyor belt maintained in tension and passing through a bore of an imaging device; and motorized pulleys disposed at opposite ends of the bore to move the conveyor belt through the bore and/or ensure continuous tension is applied to the belt. Table supports are positioned outside of the bore of the imaging device on opposite ends of the bore and support the conveyor belt outside the bore of the imaging device.

In another disclosed aspect, an image acquisition system includes a medical imaging device configured to generate imaging data for a subject disposed in an examination region; and a medical imaging subject support table including a conveyor belt maintained in tension and passing through the examination region of the medical imaging device.

In another disclosed aspect, an imaging system includes a medical imaging subject support table with a movable portion configured to move through a bore of an imaging device. Table supports are positioned outside of the bore of the imaging device on opposite ends of the bore and support the movable portion outside the bore of the imaging device. One or more support bars are disposed in the bore and connected at their ends with the table supports, the support bars providing support for the movable portion inside the bore.

In another disclosed aspect, an imaging system includes a gantry defining a bore, and a table extending through the bore. The table is configured to move the patient through the bore during an image acquisition procedure. One or more radiation absorbing (high-Z) layers are embedded into the table adjacent opposing ends of the bore. The radiation absorbing layers are configured to reduce radiation from entering an imaging area within the bore.

One advantage resides in providing an imaging system with a patient table comprising a conveyor belt that is under tension to ensure that there is adequate support and negligible sagging in an imaging field of view. The conveyor belt which serves as the patient support is held in tension rather than being a rigid support, and can therefore be made thinner.

Another advantage resides in providing an imaging device with a conveyor belt made of thin material and having minimal attenuation.

Another advantage resides in reduced patient table mass disposed in the bore of the imaging device, thereby reducing attenuation.

Another advantage resides in providing an imaging device with a field of view that is virtually unobstructed, except for a conveyor belt.

Another advantage resides in reduced out-of-field of view (FOV) radiation straying into the imaging FOV.

Another advantage resides in providing patient tables amenable to optimal design trading off patient support versus attenuating table mass disposed in the bore.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the invention.

FIG. 1 diagrammatically illustrates an imaging device with a component for a patient to lie on during a medical procedure in accordance with one embodiment.

FIG. 2 diagrammatically illustrates an imaging device with a component for a patient to lie on during a medical procedure in accordance with another embodiment.

FIG. 3 diagrammatically illustrates an imaging device with a component for a patient to lie on during a medical procedure in accordance with another embodiment.

FIG. 4 diagrammatically illustrates an imaging device with a component for a patient to lie on during a medical procedure in accordance with another embodiment.

### DETAILED DESCRIPTION

The following discloses an improved patient table in the form of a belt conveyor system with a conveyor belt on which the patient is disposed. The belt, being held in tension, can support the weight of the portion of the patient disposed in the bore without any underlying support. This allows the “table” (i.e., belt) to be around 5 mm or thinner (3 mm in an illustrative embodiment) and hence present only a few percent attenuation; as compared with a conventional cantilevered or other rigid table which is typically on the order of two inches thick and may have a complex construction such as including a carbon fiber structural shell with a low-density filler.

Some embodiments employ a closed-circuit belt that runs through the bore and has a return path passing underneath the imaging device. Such construction may be problematic since an opening must be provided between the floor and the imaging device gantry. In other embodiments, take-up rolls are provided on the two opposite ends of the conveyor so that the return path passing underneath the imaging device is eliminated. In either configuration, motorized pulleys at opposite ends of the belt path provide pulling forces to be applied during movement of the patient in either direction. In embodiments with take-up rolls, these may be the motorized pulleys, or the motorized pulleys may be separate. In general, any conveyor belt drive configuration can be employed which places the belt in tension in the gap defined by the bore.

In most imaging devices and for most imaging subjects, the belt in tension is expected to be sufficient to support the patient. However, if further support is needed it is contemplated in further variant embodiments to provide an extensible belt support that can be extended into the gap defined by the bore. As this belt support provides only a supporting role, it can be made thinner and present less attenuation as compared with the conventional rigid table. In another contemplated approach, sag of the belt across the gap defined by the bore is measured using a laser or the like, and if the sag is too great the tension may be increased to reduce the sag to an acceptable level, or the extensible belt support (if provided) may be deployed. In a more advanced implementation, if the sag measurement is quantitative then it can be used to provide feedback control to the tensioners.

In another optional aspect, a high-Z radiation absorbing material (that is, a material with atoms of high atomic number providing strong radiation absorption, e.g. lead or lead alloy materials) can be embedded in the table support proximate to the edges of the bore. This reduces stray out-of-field of view (FOV) radiation into the imaging FOV. This aspect can be used with the disclosed conveyor belt-based patient table, or with a conventional patient table employing an axially translating rigid tabletop or the like.

In another optional aspect, thin ribs can extend from table supports proximate to the imaging device bore axially across the bore. These ribs provide additional support for the portion of the conveyor belt extending through the bore. As ribs with large gaps between the ribs, the attenuation introduced is again low. This improvement also can be used with or without the disclosed conveyor belt approach.

In further embodiments, an image acquisition device includes a table support and a management system split into two parts on both sides of the imaging gantries. The two table pieces are joined only by the conveyor belt that covers the top surface of the table so that the patient can be fully transported from scan start to the scan end positions. The conveyor belt runs continuously through the gantry of the imaging device. As it supports the patient in the bore without a hardtop, the belt is kept under tension by the belt conveyor system to ensure there is no sagging caused by the weight of the patient. The patient is transported by the movement of the conveyor belt as needed to execute the imaging scan.

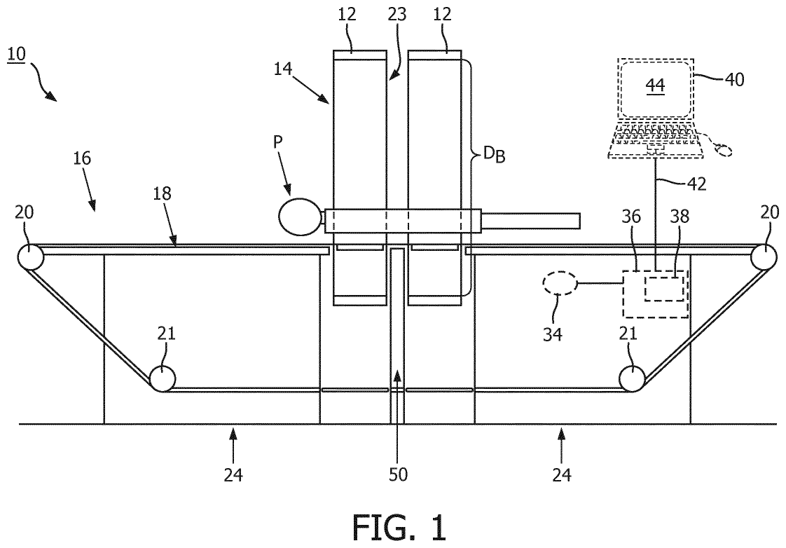

With reference to FIG. 1, an illustrative imaging device 10 for acquiring images of a patient P is shown. The imaging device 10 can be any suitable imaging device, such as an X-ray imaging device, a transmission computed tomography (CT) imaging device, a positron emission tomography (PET) imaging device, a gamma camera for single photon emission computed tomography (SPECT), a hybrid PET/CT device, a hybrid PET/magnetic resonance (MR) device, and the like. As shown in FIG. 1, the illustrative imaging device 10 is a hybrid PET/CT device that includes a gantry or medical imaging device 12 containing one or more PET detectors rings implementing the PET modality and an x-ray tube/x-ray detector panel assembly on an internal rotating gantry implementing the CT modality (internal components not shown). A bore or an examination region 14 of diameter D_{B} as indicated in FIG. 1 is defined by the gantry 12 through which a patient moves into during an image acquisition procedure. As is known in the art, PET imaging entails administering a positron-emitting radiopharmaceutical to a patient who is then disposed in the bore 14 and imaged by detecting oppositely directed 511 keV gamma rays generated by positron-electron annihilation events. The PET detector ring(s) detect coincident 511 keV gamma ray pairs defining lines of response (LORs), and a suitable PET imaging data reconstruction is applied to generate a reconstructed PET image of the radiopharmaceutical distribution in the patient. The radiopharmaceutical is usually chosen to accumulate in organs or tissue of interest thereby providing imaging of those organs/tissue, and may also provide functional imaging. In a variant known as time-of-flight (TOF) PET, the PET detectors are sufficiently high speed to further localize the source positron-electron annihilation event along the LOR. In the case of CT, the x-ray tube transmits an x-ray beam through the patient disposed in the bore 14, the x-rays are detected after transmission through the patient by an oppositely arranged x-ray detector panel. By rotating the x-ray tube and x-ray detector together on an internal rotating gantry, projection views over 360° are obtained, and the resulting x-ray projections are reconstructed to form an image of x-ray attenuation density in the patient (e.g. emphasizing bones or other tissue with higher x-ray absorption). Again, this is merely an illustrative example, and the imaging device may instead be a standalone CT imaging device, standalone PET imaging device, or some other single-modality or multi-modality imaging device such as a gamma camera or PET/CT.

The imaging device 10 also includes a belt conveyor system 16 that includes a conveyor belt 18 and at least two motorized pulleys (or drums) 20 disposed at opposite ends of the bore 14. In some embodiments, the conveyor belt 18 has a thickness of 5 mm or less so as to limit attenuation of the operative attenuation used in the imaging (e.g. the 511 keV gamma rays detected in PET imaging, or the x-rays in the case of CT). Advantageously, the conveyor belt 18 can be made thin so as to only have a few percent attenuation. The conveyor belt 18 is maintained in tension by the pulleys 20, and passes through the bore 14 without bottom support in the bore. The pulleys 20 are configured to move the conveyor belt 18 through the bore 14. The belt conveyor system 16 may include additional rollers or tensioners 21 or the like to support and tension the conveyor belt 18. The imaging device 10 includes table supports 24 located outside of the bore 14 over which the conveyor belt 18 moves. The table supports 24 provide bottom support for the conveyor belt 18 outside of the bore 14, so that the tension of the conveyor belt is required to support the belt only in the bore 14. The pulleys 20 are disposed at on opposite sides of the bore 14.

In the illustrative hybrid PET/CT device of FIG. 1, the gantry 12 is a split gantry, i.e. has separate gantries for the PET and CT modalities, with a gap 23 between them, and an optional catcher 50 provides a further support for the belt midway through the bore 14. This catcher 50 is optional and may be omitted in the split gantry design; moreover in some other embodiments a single gantry (with no split) may contain both the PET and CT imaging modalities, in which case it may not be convenient to add a catcher. In other words, the catcher 50 may be used with split gantries, non-split gantries, or omitted altogether.

Because the conveyor belt 18 is under tension, the choices of viable materials for the conveyor belt 18 is expanded compared with a rigid tabletop support. In some embodiments, the conveyor belt 18 is made of a cloth fabric or synthetic polymer fibers having low attenuation coefficient for the operative radiation.

The pair of table supports 24 are positioned underneath the portions of the conveyor belt 18 located outside of the bore 14 and extend from the ends of the bore to the respective pulleys 20. As shown in FIG. 1, the supports 24 are positioned outside of the bore 14 to support the conveyor belt 18 outside of the bore. The table supports 24 include a gap or conduit (not shown) to allow a return path for the conveyor belt 18 to pass through during movement. The gap or conduit allows the conveyor belt 18 to form a loop that passes through the bore 14 and returns underneath the bore.

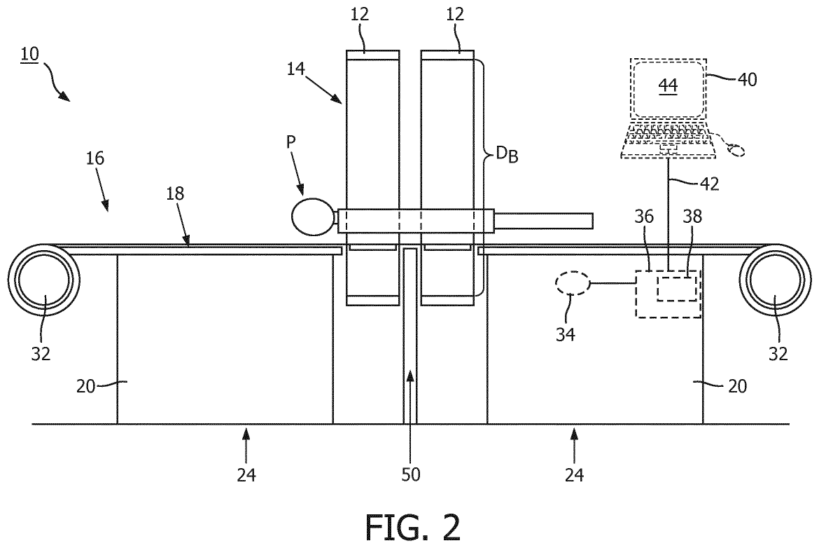

Referring now to FIG. 2, in another embodiment the belt conveyor system 16 avoids the return path of the embodiment of FIG. 1 by providing belt take up reels 32 at opposite sides of the gantry 12 are configured to take up or wind an excess portion of the conveyor belt 18 as the belt moves though the bore 14. In this embodiment, the conveyor belt 18 does not pass underneath the bore 14. Rather, the conveyor belt 18 is simply taken up by the reels 32 as the belt moves through the bore 14. The illustrative take-up reels 32 are also motor-driven so as to also serve the drive function of the pulleys 20 of the embodiment of FIG. 1 (or, said another way, the take up reels 32 are also motorized pulleys 32). However, in other embodiments separate take up reels and motorized pulleys may be provided.

With continuing reference to FIG. 2, and referring back to FIG. 1, a portion of the conveyor belt 18 that extends through the bore 14 is unsupported in the bore except by being maintained in tension by the belt conveyor system 18. For example, the pulleys 20 maintain a tension in the conveyor belt 18 by taking up or removing any slack in the conveyor belt. In another example, the take up reels 32 act as motorized pulleys to take up slack in the conveyor belt 18 to maintain the tension therein.

In some examples, the imaging system 10 may also include at least one sensor 34 configured to measure a sag value of the conveyor belt 18, or to detect sag of the conveyor belt greater than a threshold. The sensor 34 may, for example, be a laser/light detector continuity switch, arranged such that sag of the conveyor belt 18 beyond the threshold causes it to block the path from the laser to the detector of the laser/light detector switch. The imaging system 10 also includes a belt conveyor system controller 36 in communication with the at least one sensor 34. The belt system controller 36 includes at least one electronic processor 38 programmed to receive the measured sag value or sag detection from the at least one sensor 34. From the received sag value, the processor 38 of the controller 36 is programmed to increase tension of the conveyor belt 18 to reduce the sag value or to eliminate the detected sag (i.e., by controlling the motors 20 or the take up reels 32 to take up the slack in the conveyor belt). The detected sag value may also be optionally transmitted into the image reconstruction and processing software for appropriate corrections/adjustments to be made.

In other examples, a computer 40 or other electronic device including an electronic processor 42 and a display device 44 is in electrical communication with the imaging device 10. The computer 40 that includes the at least one electronic processor 42 which includes or is operatively connected to read the at least one sensor 34 and/or control the belt system controller 36. Data related to the image acquisition process can be displayed on the display device 44 of the computer 40. The at least one electronic processor 42 is operatively connected with a non-transitory storage medium that stores instructions which are readable and executable by the electronic processor 22 to perform disclosed operations including controlling the imaging device 18 to perform an imaging data acquisition process. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

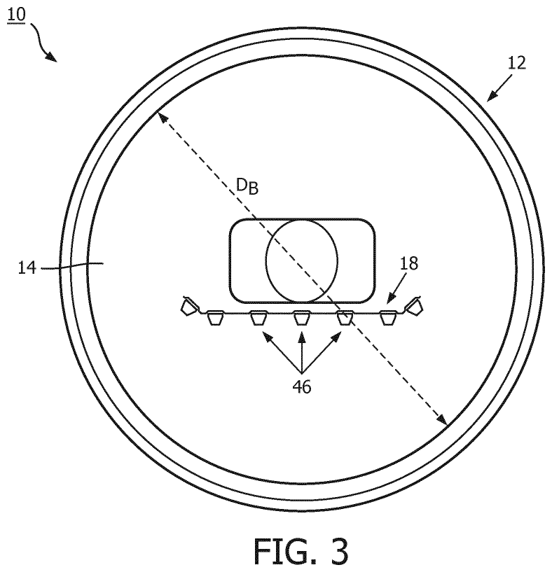

Referring now to FIG. 3, the imaging device 10 can include one or more support bars 46 that are disposed in the bore 14. The support bars 46 are connected at their ends with the table supports 24 such that they span a width of the table. The support bars 46 are configured to provide supplementary support for the conveyor belt 18 inside the bore 14. As used herein, the term “supplementary support” refers to an insufficient support to bear a full weight of the patient in the bore 14. Rather, the supplementary support provided by the support bars 46 are configured to support a portion of the patient's weight. It will be appreciated that the support bars 46 can be implemented in some embodiments of the imaging device 10 that does not include the belt conveyor system 16. The support bars 46 can also be useful when certain phantoms are to be acquired in the imaging device 12.

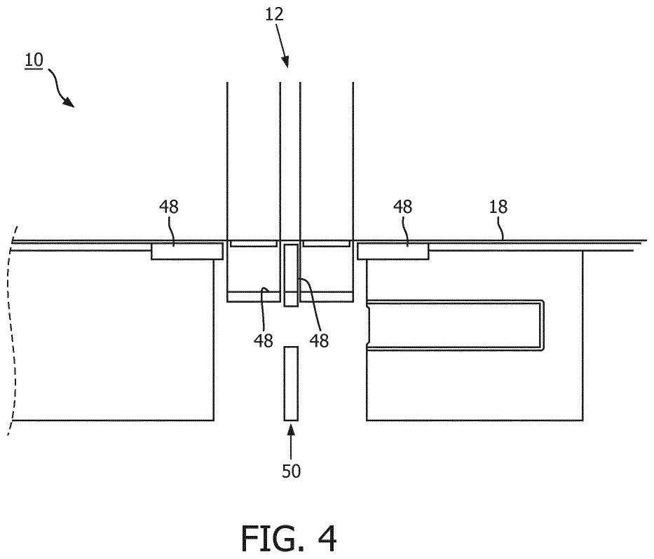

FIG. 4 shows that the imaging system 10 can include one or more radiation absorbing layers 48. The radiation absorbing layers 48 are embedded into the table supports 24 adjacent opposing ends of the bore 14. The radiation absorbing layers 48 include a high Z material, such as lead, that attenuates radiation so as to reduce, prevent, or eliminate such radiation from entering an imaging area within the bore 14. As used herein, the term “radiation-absorbing” refers to a material that absorbs or blocks gamma rays from out the FOV during the image acquisition process. The radiation absorbing layers 48 can also provide supplemental support for the patient. It will be appreciated that the radiation-absorbing layers 48 can be implemented in some embodiments of the imaging device 10 that does not include the belt conveyor system 16. These radiation absorbing layers 48 operate to block out-of-FOV radiation from entering the imaging FOV.

In some embodiments, the conveyor belt 18 does not hold the whole patient weight. This is because the axial width of the gantry 12 is usually much less than the height of the patient. Thus, the patient extends outside of the bore 14 on one or both sides. Much of the patient's weight is thus supported by the table supports 24 located outside of the bore 14.

Typically, the axial extent of the PET imaging FOV is about 18 cm. That means the tension of the conveyor belt 18 is only required to support the portion of the patient weight distribution located in that 18 cm gap. The pressure to the belt 18 caused by this portion of the human body weight distribution may be further reduced because the human body is self-sustainable for the most parts (due to the residual muscle tonus). In the following, an estimate of the belt tensioning design is described as a non-limiting illustrative example.

When imaging the extremities, such as during brain scans, there may be no support on the other side of the gantry. However, the typical weight of human head is in the range of 4.5 to 5 kg. As a result, the adequately tensioned conveyor belt 18 should be able to support it with minimum sagging by the equation:

F≈gM*L/S

where F is a tension force that needs to be applied to the conveyor belt hanging over a gap of length L in order to balance the weight M at sagging of S under gravitational acceleration g. Substituting the sample values discussed above with allowable maximum sagging of 3 mm gives the tension force applied to the belt to be equal to:

F=9.81 m/s\hat{ }2*5 kg*0.18 m/0.003 m=2943 N≈300 kgf,

which is a reasonable value and can be achieved with proper belt tensioning mechanism.

The proposed embodiments do not increase the total footprint of the imaging device, which is an important characteristics for hospitals where available area is scarce. It is correct that single side support patient tables can have very little footprint when in contracted state. However, during the patient scan it would still need to extend all the way beyond the scanner gantry, leading to so called invisible footprint that must remain clear. Therefore, in the disclosed embodiments do not increase the total effective footprint of the system.

These disclosed embodiments contribute to radioactive dose reduction to the patient, shortened scan times and/or reduced demand for crystal and electronics material by reducing the total attenuation and scatter. It is also expected that the overall image quality and quantitation will be improved due to reduced attenuating medium in the FOV of the imaging device. Also, the overall patient supporting and transportation mechanism in the disclosed embodiments can lead to reduced cost (e.g., the patient table cost can be significantly reduced as compared to the current ones that need to minimize sagging and deflection in PET/CT etc., such reduction can easily offset the cost of the conveyor belt).

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A medical imaging subject support table, comprising:
a belt conveyor system including:
a conveyor belt maintained in tension and passing through a bore of an imaging device; and
motorized pulleys disposed at opposite ends of the bore to move the conveyor belt through the bore; and
table supports positioned outside of the bore of the imaging device on opposite ends of the bore and supporting the conveyor belt outside the bore of the imaging device;
at least one sensor configured to measure a sag value of the conveyor belt or to detect sag of the conveyor belt greater than a threshold.

2. The medical imaging subject support table of claim 1, wherein:
the belt conveyor system further includes take up reels disposed at opposing ends of the bore and onto which an excess portion of the conveyor belt winds; and
wherein the conveyor belt does not pass underneath the bore.

3. The medical imaging subject support table of claim 1, wherein the conveyor belt forms a loop passing through the bore and returning underneath the bore.

4. The medical imaging subject support table of claim 1, wherein a portion of the conveyor belt extending through the bore is unsupported in the bore except by being maintained in tension by the belt conveyor system.

5. The medical imaging subject support table of claim 1, further comprising:
support bars disposed in the bore and connected at their ends with the table supports, the support bars providing supplementary support for the conveyor belt inside the bore.

6. The medical imaging subject support table of claim 5, further comprising:
one or more radiation absorbing layers embedded into the medical imaging subject support table adjacent opposing ends of the bore, the radiation absorbing layers being configured to reduce radiation from entering an imaging area within the bore.

7. (canceled)

8. The medical imaging subject support table of claim 1, further comprising:
a belt conveyor system controller comprising at least one electronic processor programmed to:
receive the measured sag value or sag detection from the at least one sensor; and
increase tension of the conveyor belt to reduce the sag value or to eliminate the detected sag.

9. (canceled)

10. An image acquisition system, comprising:
a medical imaging device configured to generate imaging data for a subject disposed in an examination region;
a medical imaging subject support table comprising a conveyor belt maintained in tension and passing through the examination region of the medical imaging device; and
at least one sensor configured to measure a sag value of the conveyor belt or to detect sag of the conveyor belt greater than a threshold.

11. The image acquisition system of claim 10, wherein the medical imaging subject support table further includes motorized pulleys disposed at opposite ends of the examination region to move the conveyor belt through the examination region.

12. The image acquisition system of claim 11, wherein the medical imaging device includes at least one of a positron emission tomography (PET) imaging device, and a transmission computed tomography (CT) imaging device, and the examination region comprises a bore.

13. The image acquisition system of claim 12, wherein:
the medical imaging subject support table further includes take up reels disposed at opposing ends of the bore and onto which an excess portion of the conveyor belt winds; and
wherein the conveyor belt does not pass underneath the bore.

14. The image acquisition system of claim 13, wherein the conveyor belt forms a loop passing through the bore and returning underneath the bore.

15. The image acquisition system of claim 12, wherein a portion of the conveyor belt extending through the bore is unsupported in the bore except by being maintained in tension by the belt conveyor system.

16. The image acquisition system of claim 12, further comprising:
support bars disposed in the bore and connected at their ends with the table supports, the support bars providing supplementary support for the conveyor belt inside the bore.

17. The image acquisition system of claim 12, further comprising:
one or more radiation absorbing layers embedded into the medical imaging subject support table adjacent opposing ends of the bore, the radiation absorbing layers being configured to reduce radiation from entering an imaging area within the bore.

18. The image acquisition system of claim 12, further including at least one sensor configured to measure a sag value of the conveyor belt or to detect sag of the conveyor belt greater than a threshold.

19. The image acquisition system of claim 18, further comprising:
a belt conveyor system controller comprising at least one electronic processor programmed to:
receive the measured sag value or sag detection from the at least one sensor; and
increase tension of the conveyor belt to reduce the sag value or to eliminate the detected sag.

20. The image acquisition system of claim 19, wherein the conveyor belt has a thickness of 5 mm or less.

21. An imaging system, comprising:
a medical imaging subject support table including:
a movable portion configured to move through a bore of an imaging device;
table supports positioned outside of the bore of the imaging device on opposite ends of the bore and supporting the movable portion outside the bore of the imaging device; and
one or more support bars disposed in the bore and connected at their ends with the table supports, the support bars providing support for the movable portion inside the bore.

22. An imaging system, comprising:
a gantry defining a bore;
a table extending through the bore, the table being configured to move the patient through the bore during an image acquisition procedure; and
one or more radiation absorbing layers embedded into the table adjacent opposing ends of the bore, the radiation absorbing layers being configured to prevent radiation from entering an imaging area within the bore.
